(19) United States
(12) Patent Application Publication
(10) Pub. No.: US 2025/0179437 A1
(43) Pub. Date: Jun. 5, 2025

(54) LATRUNCULIN A FOR PRODUCING PRIMITIVE STREAK CELLS

(71) Applicant: UNIVERSITY HEALTH NETWORK, Toronto (CA)

(72) Inventors: Maria Cristina NOSTRO, Toronto (CA); Emily Catherine MCGAUGH, Brampton (CA); Paraish Shivam MISRA, Toronto (CA)

(21) Appl. No.: 18/964,047

(22) Filed: Nov. 29, 2024

Related U.S. Application Data
(60) Provisional application No. 63/604,825, filed on Nov. 30, 2023.

(51) Int. Cl. C12N 5/071

(52) U.S. Cl. CPC C12N 5/0678; C12N 5/0689; C12N 2501/16; C12N 2501/415; C12N 2501/727; C12N 2501/999; C12N 2506/45

(57) ABSTRACT

There is described herein a method of producing primitive streak cells for differentiation to definitive endoderm from a population of human pluripotent stem cells (hPSCs), the method comprising: differentiating a population of hPSCs to primitive streak cells in culture medium comprising Latrunculin A (LatA).

### RELATED APPLICATIONS

This application claims priority to U.S. Provisional Patent Application No. 63/604,825, filed on Nov. 30, 2023, the entirety of which is incorporated herein by reference.

### FIELD OF THE INVENTION

The invention relates to the differentiation of stem cells and in particular to the use of Latrunculin A for producing primitive streak cells intended for differentiation into definite endoderm.

### BACKGROUND OF THE INVENTION

The ability to differentiate human pluripotent stem cells (hPSCs) into pancreatic endocrine cells in vitro offers an unprecedented access to islet-like cells (hPSC-islets), both for research into their function and pathology, and for the therapy of type 1 diabetes (T1D). Following the specification of hPSCs into pancreatic progenitors (hPSC-PPs), the final stages of hPSC-islet differentiation produce a variety of endocrine cell types, including mono-hormonal insulin-producing beta-like cells, which are likely the most important components of a therapeutic product for T1D. Although many pathways that promote the differentiation of hPSC-PPs into pancreatic endocrine cells have been identified, failure to successfully generate definitive endoderm at the early steps of differentiation often leads to poor outcomes at the end stages and increases the likelihood of generating cell products containing off-target contaminating cells.

### SUMMARY OF THE INVENTION

There is provided herein a description of studies designed to understand mechanisms regulating primitive streak induction and definitive endoderm commitment. The present studies found that Latrunculin A-treated hPSCs generated primitive streak cells at high efficiencies.

In an aspect, there is provided a method of producing primitive streak cells for differentiation to definitive endoderm from a population of human pluripotent stem cells (hPSCs), the method comprising: differentiating a population of hPSCs to primitive streak cells in culture medium comprising Latrunculin A (LatA).

In an aspect, there is provided the population of primitive streak cells produced by the method described herein.

In an aspect, there is provided the population of definitive endoderm cells produced by the method described herein.

In an aspect, there is provided the population of progenitor cells produced by the method described herein. In some embodiments, the progenitor cells are pancreatic progenitor cells.

In an aspect, there is provided the population of progenitor cells produced by the method described herein. In some embodiments, the progenitor cells are lung progenitor cells.

In an aspect, there is provided a use of the population described herein for cell therapy in a subject in need thereof.

In an aspect, there is provided a use of the population described herein for cell therapy in a subject in need thereof.

In an aspect, there is provided a use of the population described herein for the screening of therapeutic agents.

In an aspect, there is provided a use of the population described herein to investigate the effect of one or more therapeutic agents, diagnostic agents and/or conditions on said population or said population's development or differentiation.

### BRIEF DESCRIPTION OF FIGURES

These and other features of the preferred embodiments of the invention will become more apparent in the following detailed description in which reference is made to the appended drawings wherein:

FIG. 1: Summary of the application of Latrunculin A. Schematic describing the sequence of growth factors, signaling compounds and small molecules applied during hPSC-islet differentiation to definitive endoderm.

FIG. 2: Latrunculin A improved differentiation of hPSCs into primitive streak and subsequently definitive endoderm. Latrunculin A improves differentiation into primitive streak and definitive endoderm a) Light microscopy image of hPSCs cultured at low and high density prior to differentiation. b) Representative flow cytometry plots for BRA and SOX2 expression at 24 hours after the initiation of differentiation. c) Quantification of BRA+ cells at 24 hours after the initiation of differentiation. One-way ANOVA followed by Holm-Sidak correction; * p<0.05. d-e) Representative flow cytometry plots (d) and quantification of day 3 cells generated with and without latrunculin A for definitive endoderm markers SOX17 and FOXA2 (e) and CXCR4 and cKIT (f). * p<0.05, ** p<0.01. All error bars represent the standard error of the mean. Legend: CTRL, control; LatA, Latrunculin A; ns, non-significant.

FIG. 3: Latrunculin A-derived primitive streak improves differentiation to pancreatic and lung lineages. a) Schematic describing the differentiation of hPSC-to pancreatic and lung derivatives in the absence of MEFs, with or without Latrunculin A. b-e) Representative flow cytometry plots and quantification of pancreatic progenitor markers PDX1 and NKX6-1 (b-c) and beta-like cells markers C-peptide (CPEP) and NKX6-1 (d-e) with and without Latrunculin A. f) Representative flow cytometry plots for NKX2-1 after lung differentiation. g) Quantification of NKX2-1+ cells at day 13. Paired t-test; * p<0.05. h) Immunofluorescence of control and LatA-treated cells differentiated to NKX2-1+ lung progenitors. Paired t-test; * p<0.05, ** p<0.01. All error bars represent the standard error of the mean. Legend: CTRL, control; LatA, Latrunculin A.

### DETAILED DESCRIPTION

In the following description, numerous specific details are set forth to provide a thorough understanding of the invention. However, it is understood that the invention may be practiced without these specific details.

In an aspect, there is provided a method of producing primitive streak cells for differentiation to definitive endoderm from a population of human pluripotent stem cells (hPSCs), the method comprising: differentiating a population of hPSCs to primitive streak cells in culture medium comprising Latrunculin A (LatA).

The term “pluripotent stem cell” as used herein refers to a cell that has the capacity to self-renew by dividing, and to develop or differentiate, under different conditions, to more than one differentiated cell type, for example into one or more cell types characteristic of the three germ cell layers, and includes embryonic stem cells and induced pluripotent stem cells. Embryonic stem cells and induced pluripotent stem cells are examples of pluripotent stem cells. Pluripotent cells are characterized by their ability to differentiate to more than one cell type using, for example, a nude mouse teratoma formation assay. Pluripotency is also evidenced by the expression of embryonic stem (ES) cell markers.

As used herein, “primitive streak” refers to the structure that appears in the posterior region of the gastrulating embryo where the epiblast cells undergo 4pithelia-mesenchymal transition and ingressional movement to form the germ layers. In human development, this occurs at approximately two weeks after fertilization.

As used herein, “definitive endoderm” refers to one of the three primordial germ cell layers that can be generated from human pluripotent stem cells and which collectively can give rise to all the cell types of the post-natal human body.

In the context of a cell, the term “differentiated”, or “differentiating” is a relative term and a “differentiated cell” is a cell that has progressed further down the developmental pathway than the cell it is being compared with, and in some cases a “differentiated cell” is more specialized. In some cases, a “differentiated cell” is a cell that changed from one cell type to another. Thus, stem cells can differentiate to lineage-restricted precursor cells (such as an endodermal progenitor cell), which in turn can differentiate into other types of precursor cells further down the pathway and then to an end-stage differentiated cell, which plays a characteristic role in a certain tissue type, and may or may not retain the capacity to proliferate further.

The term “cell culture medium” (also referred to herein as a “culture medium” or “medium”) as referred to herein is a medium for culturing cells containing nutrients that maintain cell viability and support proliferation and optionally differentiation. The cell culture medium may contain any of the following in an appropriate combination: salt(s), buffer(s), amino acids, glucose or other sugar(s), antibiotics, serum or serum replacement, and other components such as peptide growth factors, vitamins etc. Cell culture media ordinarily used for particular cell types are known to those skilled in the art.

The term “contacting” refers to any suitable means by which the cell is cultured or incubated with the component(s) together in vitro. For example, the compound is added to the cells in culture, or is transferred to or mixed with culture medium containing the compound. For example the cells may be treated in adherent culture, or in suspension culture, the components can be added temporally substantially simultaneously (e.g. together in a cocktail) or sequentially (e.g. within 1 hour from an addition of a first component). The cells can also be contacted with another agent such as a growth factor or other differentiation agent or environments to stabilize the cells, or to differentiate the cells further and include culturing the cells under conditions known in the art for example for culturing the pluripotent (and/or differentiated) population.

In some embodiments, the LatA is present in the culture medium on day 0.

In some embodiments, the culture medium further comprises Activin A.

In some embodiments, the culture medium further comprises a GSK3A/B inhibitor/canonical Wnt activator. Preferably, the GSK3A/B inhibitor/canonical Wnt activator is, GSK Inhibitor IX, LiCl, WNT3 or CHIR99021, further preferably CHIR99021.

In some embodiments, the culture medium further comprises a ROCK inhibitor. Preferably, the ROCK1/2 inhibitor is Y-27632, fasudil, K-115 or AR-13503, GSK269962, H-1152, RKI-1447, HA-1100, or Thiazovivin, further preferably Y-27632.

In some embodiments, the culture medium is mouse embryonic fibroblast free (MEF-free).

In some embodiments, the primitive streak cells are further differentiated into definitive endoderm cells.

In some embodiments, the differentiation into definitive endoderm is performed in the presence of Activin.

In some embodiments, the definitive endoderm cells are further differentiated into pancreatic progenitors.

The term “progenitor cell” refers to cells that have a cellular phenotype that is at an earlier step along a developmental pathway or progression than is a fully differentiated cell relative to a cell which it can give rise to by differentiation. Progenitor cells can give rise to multiple distinct differentiated cell types or to a single differentiated cell type, depending on the developmental pathway and on the environment in which the cells develop and differentiate.

The term “pancreatic progenitor cell” refers to a cell which is capable of forming any of: pancreatic endocrine cells (such as glucagon producing alpha cells, insulin producing beta cells, somatostatin producing delta cells, ghrelin producing epsilon cells and pancreatic polypeptide producing cells), or pancreatic acinar cells (e. g. amylase producing- and/or trypsin producing-pancreatic cells) or pancreatic ductal cells. Formation or development of one more of pancreatic endocrine cells, pancreatic acinar cells or pancreatic ductal cells may be induced by addition of components such as a combination of a thyroid hormone, alk5i, retinoic acid, BMP and SHH inhibition and/or resulting from the environment in which the progenitor cell develops. Similarly injection of PDX1+/NKX6-1+ pancreatic progenitor cells in vivo can result in development of endocrine, acinar and ductal cells.

As used herein, the term “insulin producing cell” refers to a cell differentiated from a pancreatic progenitor, which secretes insulin. An insulin producing cell includes functional pancreatic beta-cells, as well as pancreatic beta-like cells that synthesize, express, or secrete insulin in a constitutive or inducible manner. A population of insulin producing cells, e.g. produced by differentiating endodermal cells to pancreatic progenitors and subsequent differentiation into insulin producing cells according to the methods described herein, can be functional pancreatic beta-cells or beta-like cells (e.g., cells that have at least two characteristics of an endogenous functional beta-cell). It is also contemplated that the population of insulin producing cells, e.g. produced by the methods as disclosed herein, can comprise pancreatic beta-cells or pancreatic beta-like cells, and can also contain non-insulin producing cells (e.g. cells with a beta-cell like phenotype with the exception that they do not produce or secrete insulin).

In some embodiments, the definitive endoderm cells are further differentiated into lung progenitors.

In some embodiments, the definitive endoderm cells are further differentiated into liver progenitors.

In some embodiments, the definitive endoderm cells are further differentiated into intestine progenitors.

In some embodiments, the definitive endoderm cells are further differentiated into gastric progenitors.

In some embodiments, the definitive endoderm cells are further differentiated into esophageal progenitors.

In some embodiments, the definitive endoderm cells are further differentiated into thymus progenitors.

In some embodiments, the definitive endoderm cells are further differentiated into thyroid progenitors.

In an aspect, there is provided the population of primitive streak cells produced by the method described herein.

In an aspect, there is provided the population of definitive endoderm cells produced by the method described herein.

In an aspect, there is provided the population of progenitor cells produced by the method described herein. In some embodiments, the progenitor cells are pancreatic progenitor cells.

In an aspect, there is provided a use of the population described herein for cell therapy in a subject in need thereof.

The term “subject” or “patient” as used herein includes all members of the animal kingdom including mammals, and suitably refers to humans.

As used herein, “cell therapy” may refer to any therapy in which viable cells are injected, grafted or implanted into a patient in order to effectuate a medicinal effect. For example, by transplanting insulin producing cells into a patient as a treatment option for diabetes.

In an aspect, there is provided a use of the population described herein for cell therapy in a subject in need thereof.

In an aspect, there is provided a use of the population described herein for the screening of therapeutic agents.

In an aspect, there is provided a use of the population described herein to investigate the effect of one or more therapeutic agents, diagnostic agents and/or conditions on said population or said population's development or differentiation.

The term “treatment” as used herein as applied to a subject, refers to an approach aimed at obtaining beneficial or desired results, including clinical results and includes medical procedures and applications including for example pharmaceutical interventions, surgery, radiotherapy and naturopathic interventions as well as test treatments for treating diabetes. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, remission from a disease state, amelioration or palliation of the disease state, and remission (whether partial or total), whether detectable or undetectable. “Treatment” can also mean prolonging survival as compared to expected survival if not receiving treatment.

As used herein, “pharmaceutically acceptable carrier” means any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent.

As used herein, “therapeutically effective amount” refers to an amount effective, at dosages and for a particular period of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

The advantages of the present invention are further illustrated by the following examples. The examples and their particular details set forth herein are presented for illustration only and should not be construed as a limitation on the claims of the present invention.

### EXAMPLES

### Methods and Materials

### Human Pluripotent Stem Cell Differentiation

Human pluripotent stem cell (hPSC) lines: For protocol optimization, the H1 hPSC line¹, purchased from WiCell Research Institute (Madison, USA) was employed. This work has received approval from the Stem Cell Oversight Committee (SCOC) as the focus of a Canadian Institutes of Health Research (CIHR) Canada Graduate Scholarship-Doctoral (CGS-D) award.

hPSC expansion and passage: Cells were cryopreserved in a media composed of 50% IMDM (Life Technologies #12440) or StemMACS iPS-brew XF (Miltenyi Biotec #130-104-368), 40% Fetal Bovine Serum (FBS, Gibco #12483-020), and 10% DMSO (Millipore Sigma #D2650). Cryopreserved hPSCs were thawed in DMEM:F12 (Wisent #319-085-CL) at a ratio of 1 mL cells: 9 mL media, centrifuged at 1000 revolutions per minute (RPM) for 5 minutes, and resuspended in StemMACS iPS-brew XF with 10 μM ROCK1/2 inhibitor (Y-27652, Tocris Biosciences #1254). Cells were maintained in monolayer culture in StemMACS iPS-brew XF media with daily media changes. Upon reaching full confluency, cells were passaged by single cell dissociation using TrypLE (Life Technologies #12605) with 1% DNase 1 mg/mL (Calbiochem 260913), centrifuged for 5 min at 1000 RPM, and resuspended in StemMACS iPS-brew XF with 10 μM ROCK1/2 inhibitor (Y-27652, Tocris Biosciences #1254). Cells were split at a cell line-specific ratio to ensure confluency within 2-3 days, upon which differentiation was initiated.

hPSC differentiation: On day 0, confluent cultures of hPSCs in monolayer format were washed with Calcium- and Magnesium-free dPBS (Corning #20-031-CV) and fed with media with or without Latrunculin A to generate primitive streak (day 1) and definitive endoderm (day 3). The media used at day 0, 1, and 2 are specified in the tables below (Tables 1.1-1.2). When reagents were obtained from suppliers as liquids, the concentration is provided as a percentage of reagent volume/base media volume. When reagents were provided as desiccated powders that required reconstitution, the concentration is specified in either mass or molarity per unit volume of fully reconstituted media. All media were supplemented with 1% vol/vol Gibco Penicillin-Streptomycin (ThermoFisher Scientific #15070063).

TABLE 1.1
Media used at day 0

| | | | | Catalogue |
|---|---|---|---|---|
| Reagent | Concentration | | Supplier | number |
| RPMI | Base media | | Wisent | 350-000-CL |
| Gibco Bicarbonate | 2% | (v/v) | ThermoFisher | 25080094 |
| | | | Scientific | |
| Gibco Non- | 1% | | ThermoFisher | 11140050 |
| essential amino | | | Scientific | |
| acids | | | | |
| B27 ™ supplement, | 1% | (v/v) | ThermoFisher | A1895601 |
| minus insulin | | | Scientific | |
| Hyclone Glutamine | 1% | (v/v) | Fisher scientific | SH30034.01 |
| Recombinant | 100 | ng/mL | Bio-techne | 338-AC |
| human Activin A | | | | |
| CHIR99021 | 3 | μM | Tocris Biosciences | 1254 |
| Y-27652 | 10 | μM | Sigma | T6397 |
| Latrunculin A* | 0.3% | (v/v) | Cayman Chemical | 10010630 |

Legend: v/v (volume/volume); *Used in select experiments as indicated in text and figures.

TABLE 1.2
Media used at day 1 and day 2

| | | | | Catalogue |
|---|---|---|---|---|
| Reagent | Concentration | | Supplier | number |
| RPMI | Base media | | Wisent | 350-000-CL |
| Gibco Bicarbonate | 2% | (v/v) | ThermoFisher | 25080094 |
| | | | Scientific | |
| Gibco Non- | 1% | | ThermoFisher | 11140050 |
| essential amino | | | Scientific | |
| acids | | | | |
| B27 ™ supplement, | 1% | (v/v) | ThermoFisher | A1895601 |
| minus insulin | | | Scientific | |
| Hyclone Glutamine | 1% | (v/v) | Fisher scientific | SH30034.01 |
| Recombinant | 100 | ng/mL | Bio-techne | 338-AC |
| human Activin A | | | | |

Following definitive endoderm formation, cells were treated with differentiation media to generate either pancreatic or lung cells.

In an embodiment, the method comprises steps for differentiate the endodermal cell population to pancreatic cells. For example, as described herein differentiation from a an endodermal cell at day 3 differentiation to pancreatic lineages involves a series of steps that will differentiate cells to pancreatic progenitors cells at day 12-13 as described by Nostro et al 2015² or Yung and Poon 2019³ and endocrine cells from day 20 onwards as described by Rezania et al 20144, Pagliuca et al., 2014⁵, Hogrebe et al. 2020⁶ or Balboa et al., 2022⁷, where typically stage 1 is known to generate definitive endoderm, stage 4 is known to generate pancreatic progenitors and stage 6 is known to generate islet-like cells. Stage 2 can span for example 2 to 3 days and stage 3 can span for example 1 to 4 days. Stage 2 for example can include contacting the population to be differentiated (e.g. the Stage 1 endoderm cells) with Fibroblast Growth Factor 10 (FGF10), optionally wingless-type MMTV integration site family member 3A (Wnt3a), optionally Retinoic acid (RA), optionally Activin A, and optionally Dorsomorphin to produce Stage 2 differentiated cells. Stage 3 for example can include contacting the population to be differentiated (e.g. the Stage 2 differentiated cells) with Noggin, Sant-1 (or any other hedgehog (HH) signaling inhibitors), Retinoic acid (RA), optionally FGF10 to provide an endodermal cell population. Dorsomorphin can be substituted for example with other noggin components such as chordin LDN 193189 and BMPRs; ActA can be substituted with other Nodal agonists and Wnt 3a can be substituted with other Wnt signaling agonists such as or other wnt/beta catenin agonist such as CHIR99021. Similarly, bFGF and FGF10 can be substituted with other FGFs or compounds that activate the same receptor as bFGF or FGF10 respectively. Retinoic acid can be substituted for example by a retinoic acid analog. The protocol can for example be the protocol previously described². Protocols for generating Stage 3 cells are described for example in U.S. Pat. Nos. 7,989,204, 7,993,916, 8,129,182 and 8,187,878 each of which are incorporated by reference.

Stage 4 for example can include contacting the population to be differentiated (e.g. the Stage 3 differentiated cells) with Noggin, Epidermal Growth factor (EGF), Nicotinamide, optionally Sodium Butyrate to provide an endodermal cell population expressing PDX1 and NKX6-1. Nicotinamide can be substituted for example with other components such as tankyrase inhibitors WIK14 as described³. Protocols for generating Stage 4 cells are described for example in X Patents (list our two patents) each of which are incorporated by reference^{2,3}.

Differentiation of PDX1/NKX6-1 double positive progenitor cells into endocrine cells is performed by, for example, methods previously disclosed⁴⁻⁷. Stage 5 for example can include contacting the population to be differentiated (e.g. the Stage 4 differentiated cells) with 3,3′,5-Triiodo-L-thyronine (T3), LDN193189 (LDN), Repsox, Dibenzazipine, optionally betacellulin, optionally retinoic acid (RA), optionally latrunculinA, optionally Y-27652 to provide an endocrine cell population expressing Chromogranin A and NKX2-2. LDN 193189 can be substituted for example with other noggin components such as chordin Dorsomorphin and BMPRs. Dibenzazipine can be substituted for example with other gamma secretase inhibitor components such as (2S)—N-[(3,5-Difluorophenyl)acetyl]-L-alanyl-2-phenyl]glycine 1,1-dimethylethyl ester (DAPT), Retinoic acid can be substituted for example by a retinoic acid analog.

Differentiation of endocrine cells into islet-like cells is performed by, for example, the methods previously disclosed⁴⁻⁷. Stage 6 for example can include contacting the population to be differentiated (e.g. the Stage 5 differentiated cells) with 3,3′,5-Triiodo-L-thyronine (T3), LDN193189 (LDN), Repsox, to provide an endocrine cell population expressing C-peptide and NKX6-1.

In an embodiment, the method comprises steps for differentiate the endodermal cell population to lung cells. For example, as described herein differentiation from an endodermal cell at day 3 of differentiation to lung lineages involves a series of steps that will differentiate cells to anterior foregut cells at day 6 and lung progenitors cells at day 14 as described by Jacob 2017⁸ and Hurley 2020⁹. The differentiation of definitive endoderm to anterior foregut cells can span for example 2 to 3 days and the differentiation from anterior foregut to lung progenitors can span for example 9 to 11 days. The differentiation of definitive endoderm to anterior foregut cells for example can include contacting the population to be differentiated (e.g. the endoderm cells generated with or without LatA) with SB431542, Dorsomorphin and Y-27652 to provide an anterior foregut population expressing SOX2.

Differentiation of anterior foregut cells into lung progenitors is performed by, for example, the methods previously disclosed^{8,9}. The differentiation from anterior foregut to lung progenitors for example can include contacting the population to be differentiated (e.g. the SOX2+ anterior foregut cells) with CHIR99021, retinoic acid (RA), recombinant human BMP4 to provide lung progenitors expressing NKX2-1.

### Flow Cytometry

For profiling of hPSC-derived cells, cells were incubated with TrypLE Express with DNase at 10 μL/mL (1%) for 3-10 minutes at 37° C., then gently dissociated with a P1000 Pipetman. An equal volume of staining buffer containing 10% heat-inactivated Gibco FBS (ThermoFisher Scientific #12483-020) and 90% PBS without Calcium and Magnesium (Corning #21-040), supplemented with DNase (100 μL/mL) was added to stop the dissociation reaction.

Dissociated cells in suspension culture were filtered through a 35 micrometer cell strainer, centrifuged, and the supernatant was discarded. All centrifuge steps were done at 930G for 2 minutes, and all antibody staining was performed in U-bottom 96-well plates. Cells were analyzed on a BD LSRFortessa flow cytometer. For multicolor experiments, compensation beads were used with fluophore-conjugated antibodies to calculate compensation parameters. Experimental data was analyzed using FlowJo Software (BD).

Cell-surface flow cytometry of hPSC-derived cells: Dissociated cells were directly incubated with directly conjugated primary antibodies (table 1.3) in staining buffer for 20 minutes at room temperature. Cells were then washed once, and resuspended in staining buffer containing 1:100,000 DAPI (Biotium #40043) and directly analyzed by flow cytometry. All washes and antibody dilutions were performed with staining buffer.

Intracellular flow cytometry of hPSC-derived cells: Cells were incubated with Zombie Violet Fixable Viability dye (diluted at 1 μL/mL in PBS without Calcium and Magnesium) for 20 minutes at room temperature. Cells were centrifuged, and then resuspended with Cytofix/Cytoperm (BD #555722) solution for 20 minutes at room temperature. Cells were centrifuged and resuspended in primary antibody solution (table 1.4) for at least 1 hour at room temperature (most antibodies) or overnight at 4° C. (any solution containing anti-human NKX6.1 primary antibody). Cells were washed once, resuspended in secondary antibody solution (table 1.5), and left to incubate for 20 minutes at room temperature. Cells were then washed once and resuspended in staining buffer for analysis. All washes and antibody dilutions were performed with Perm/Wash solution (10% BD Perm/Wash 10× stock solution, 90% PBS without Calcium and Magnesium). Due to cross-reactivity of donkey anti-mouse secondary antibodies against rat primary antibodies, solutions containing rat primary antibodies were always added after incubations with mouse primary antibodies and anti-mouse secondary antibodies were completed.

TABLE 1.3
Primary antibodies used for (live) cell-surface flow cytometry

| Species | Target | Fluorophore | Dilution | Vendor | Catalogue |
|---|---|---|---|---|---|
| Mouse | CD117 | PE | 1:100 | Invitrogen | CD11704 |
| Mouse | CD184 | APC | 1:200 | BD | 555976 |
| | | | | Pharmingen | |

TABLE 1.4
Primary antibodies used in flow cytometry

| | | | | | |
|---|---|---|---|---|---|
| Species | Target | Fluorophore | Dilution | Vendor | Catalogue |

| | | | | |
|---|---|---|---|---|
| Goat | Brachyury | 1:500 | R&D Systems | AF2085 |
| Rat | C-Peptide | 1:40 | DSHB | GN-ID4 |
| Rabbit | FOXA2 | 1:2000 | Abcam | Ab40874 |
| Rabbit | NKX2-1 | 1:500 | Abcam | Ab76013 |
| Mouse | NKX6-1 | 1:1000 | DSHB | F55A10 |
| Goat | PDX1 | 1:100 | R&D Systems | AF2419 |
| Rabbit | SOX2 | 1:300 | Cell Signaling | 3579 |
| | | | Technologies | |
| Goat | SOX17 | 1:2000 | Biotechne | AF1924 |

Legend: FC, flow cytometry; IF, immunofluorescence

TABLE 1.5
Secondary antibodies used in intracellular flow cytometry.

| Species | Target | Fluorophore | Dilution | Vendor | Catalogue |
|---|---|---|---|---|---|
| Donkey | Mouse | Alexa647 | 1:400 | Invitrogen | A31571 |
| Donkey | Rabbit | Alexa647 | 1:2000 | Invitrogen | A31573 |
| Donkey | Goat | Alexa488 | 1:400 | Jackson | 705-546- |
| | | | | ImmunoResearch | 147 |
| | | | | Laboratories Inc. | |
| Goat | Rat | PE | 1:400 | BD Pharmingen | 550767 |

Legend: FC, flow cytometry; IF, immunofluorescence

### Immunofluorescence

Sample preparation: Monolayer cells were washed with dPBS without calcium and magnesium, and fixed with paraformaldehyde (Electron Microscopy Sciences #15710-S, diluted down to 4% using dPBS without calcium and magnesium) for 20 minutes at room temperature. Cells were again washed with dPBS twice, and subsequently permeabilized using a 0.5% Triton-X (Millipore Sigma #T9284) diluted in dPBS without magnesium and calcium.

Sample antibody labelling: Samples were first incubated with blocking solution containing 10% donkey serum and 2% BSA at room temperature for 30-45 minutes. Cells were washed with dPBS without calcium and magnesium once, and then incubated with primary antibodies in a buffer containing 0.05% Triton-X 100 and 2% BSA, overnight at 4° C. Samples were then washed with dPBS without calcium and magnesium twice, and incubated with secondary antibodies in a buffer containing 0.05% Triton-X 100 and 2% BSA at room temperature for 45 minutes. Cells were washed with dPBS without calcium or magnesium once, and then incubated with DAPI (1:100,000 in dPBS without calcium and magnesium) for 20 minutes. NKX2-1 antibody was purchased from Abcam (Ab76013) and used at 1:300 dilution. Secondary donkey anti rabbit antibody was purchased from Invitrogen (A31573) and used at 1:800 dilution. Cells were imaged on an EVOS microscope (ThermoFisher Scientific).

### Statistical Analysis and Illustrations

Graphpad Prism (v 9.1.4) was used for statistical analysis and data visualization. Statistical tests employed are indicated in the figure captions.

### Results and Discussion

### Improving Specification of Definitive Endoderm Under Mouse Feeder-Independent Conditions

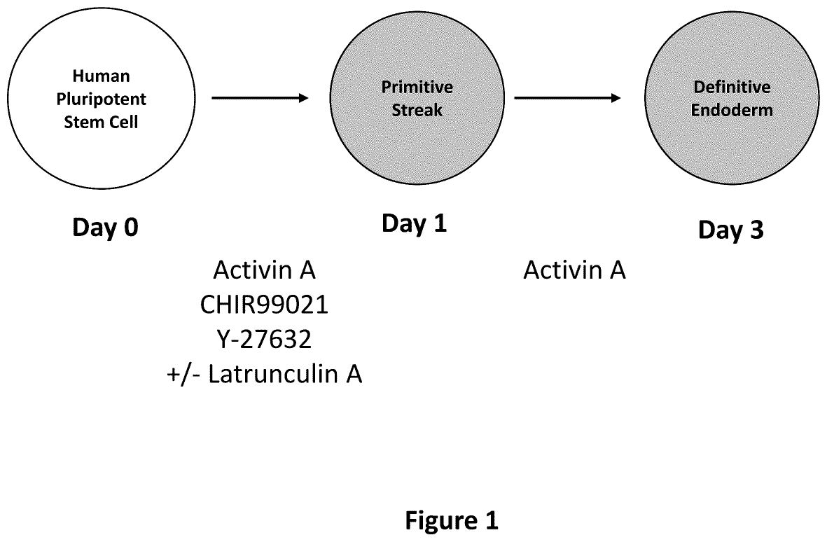

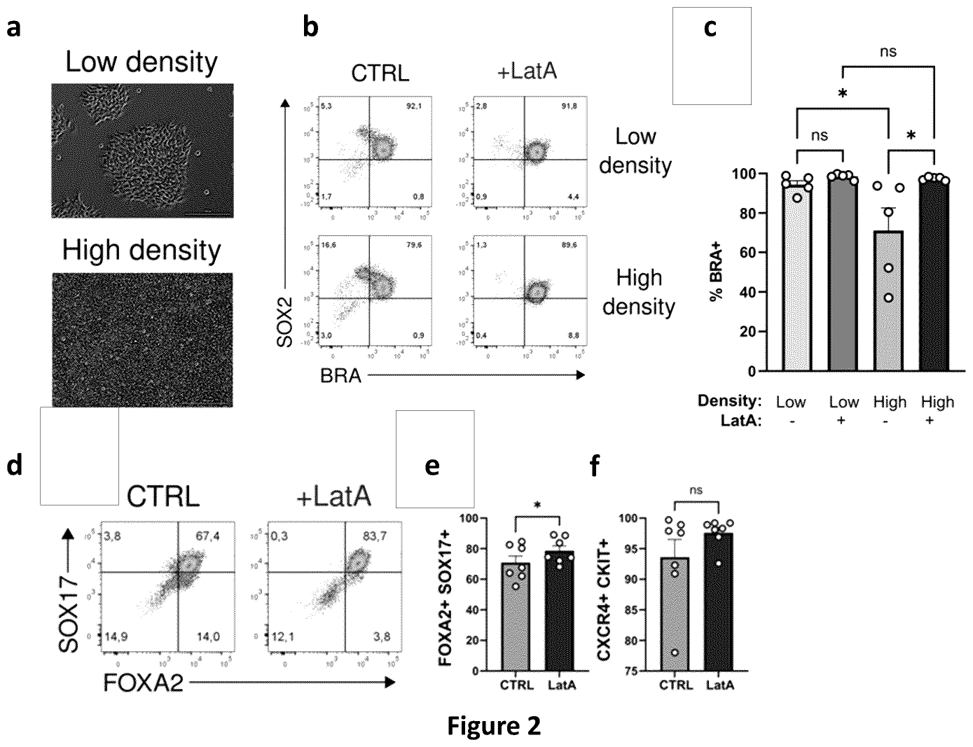

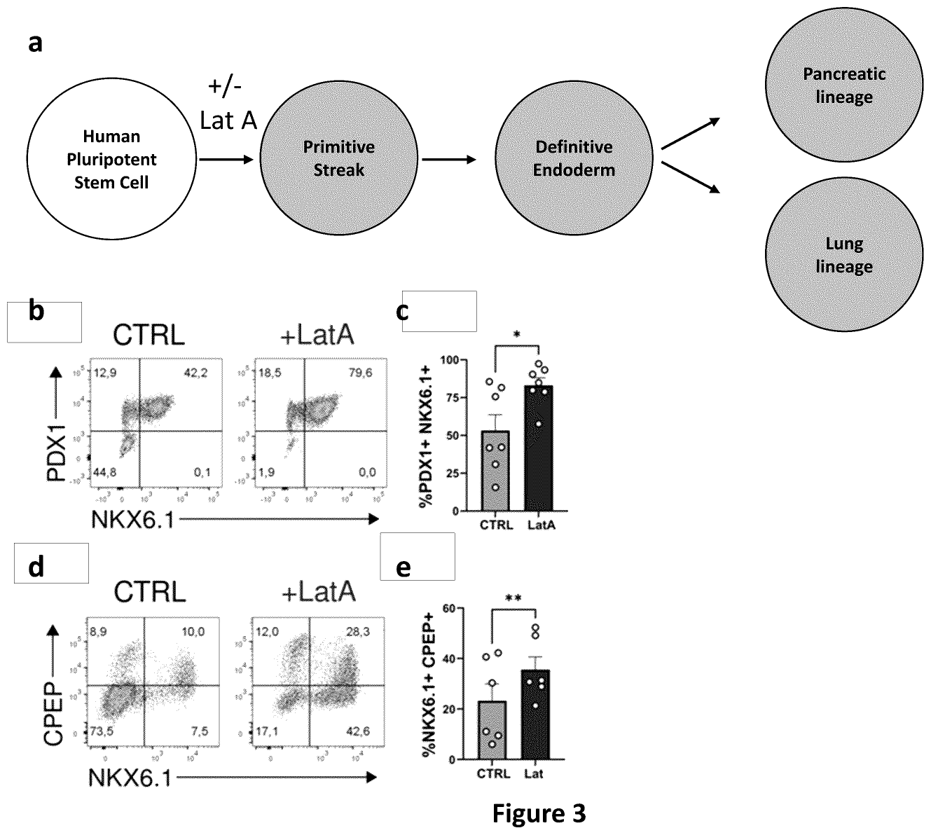

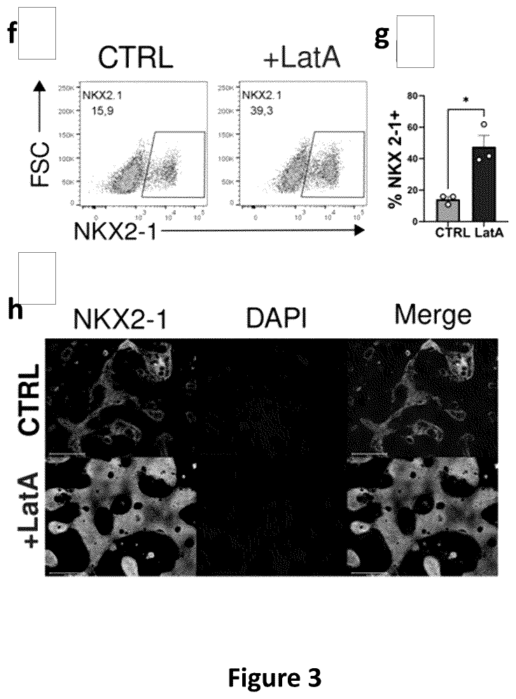

To start developing a cell product in the absence of xenogeneic sources, we first transitioned the maintenance of hPSCs to a MEF-free system using commercially-available hPSC maintenance media (FIG. 1). In this MEF-free system, the differentiation of hPSCs into Brachyury (BRA)-expressing primitive streak-like cells (PS), the first step in the differentiation of hPSCs into definitive endoderm cells, was highly influenced by the confluency of plated cells (FIG. 2a-c), as had been previously described^{10,11}. Recently, increased density of hPSCs was found to inhibit Wnt signaling, an effect which was significantly mitigated by cytoskeletal disruption¹². We thus wondered whether density-mediated Wnt inhibition was limiting our differentiation into primitive streak, since our first day of differentiation included a Wnt agonist. To determine then whether cytoskeleton disruption might mitigate the deleterious effect of high density on primitive streak differentiation, we cultured hPSCs at low and high densities, with or without the actin destabilizer Latrunculin A (LatA) on the first day of differentiation. In contrast to hPSCs grown at low density, high-density cells exhibited a notable impairment in their ability to generate BRA+ cells (FIG. 2b-c). This effect was completely abrogated with LatA treatment at day 0, yielding nearly pure populations of BRA+ primitive streak cells 24 hours after starting differentiation (FIG. 2b-c). To determine whether this resulted in improved pancreatic commitment, we followed the phenotype of high-density hPSCs with and without LatA treatment at definitive endoderm, the pancreatic progenitor and islet-like cell stages. LatA-treated hPSCs generated similar frequencies of CXCR4+c-KIT+ cells, but significantly higher frequencies of FOXA2+SOX17+ definitive endoderm (DE)-like cells at day 3 of differentiation (FIG. 2d-f), as well as a greater frequency PDX1+NKX6.1+ hPSC-derived pancreatic progenitors at day 13 along with greatly reduced PDX1−NKX6-1− cells (FIG. 3a-c). By day 23 of differentiation, LatA-treated cells displayed a greater frequency of NKX6-1+CPEP+ beta-like cells compared to the control protocol (FIG. 3d-e).

Recently, DE-like cells derived from hPSCs were found to exhibit biases with regards to their ability to generate specific endodermal lineages¹³. To determine whether LatA generally improved DE differentiation (and thereby competency to form multiple endodermal derivatives) or specifically promoted pancreatic commitment, we applied a protocol for the generation of NKX2-1+ lung progenitors^{8,9} (should include Jacob 2017 the original paper for this protocol) to DE obtained from control and LatA-treated hPSCs (FIG. 3a). LatA-treated hPSCs generated much higher frequencies of NKX2-1+ lung progenitors compared to definitive endoderm generated in the absence of LatA, suggesting that LatA has the ability to generate anterior foregut and does not bias definitive endoderm to the posterior foregut derivatives (FIG. 3.2f-h).

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

### REFERENCES

ADDIN EN.REFLIST 1 Thomson, J. A. et al. Embryonic stem cell lines derived from human blastocysts. Science 282, 1145-1147, doi:10.1126/science.282.5391.1145 (1998).
2 Nostro, M. C. et al. Efficient generation of NKX6-1+ pancreatic progenitors from multiple human pluripotent stem cell lines. Stem Cell Reports 4, 591-604, doi:10.1016/j.stemcr.2015.02.017 (2015).
3 Yung, T. et al. Sufu- and Spop-mediated downregulation of Hedgehog signaling promotes beta cell differentiation through organ-specific niche signals. Nat Commun 10, 4647, doi:10.1038/s41467-019-12624-5 (2019).
4 Rezania, A. et al. Reversal of diabetes with insulin-producing cells derived in vitro from human pluripotent stem cells. Nat Biotechnol 32, 1121-1133, doi:10.1038/nbt.3033 (2014).
5 Pagliuca, F. W. et al. Generation of functional human pancreatic beta cells in vitro. Cell 159, 428-439, doi:10.1016/j.cell.2014.09.040 (2014).
6 Hogrebe, N. J., Augsornworawat, P., Maxwell, K. G., Velazco-Cruz, L. & Millman, J. R. Targeting the cytoskeleton to direct pancreatic differentiation of human pluripotent stem cells. Nat Biotechnol, doi:10.1038/s41587-020-0430-6 (2020).
7 Balboa, D. et al. Functional, metabolic and transcriptional maturation of human pancreatic islets derived from stem cells. Nat Biotechnol 40, 1042-1055, doi:10.1038/s41587-022-01219-z (2022).
8 Jacob, A. et al. Differentiation of Human Pluripotent Stem Cells into Functional Lung Alveolar Epithelial Cells. Cell Stem Cell 21, 472-488 e410, doi:10.1016/j.stem.2017.08.014 (2017).
9 Hurley, K. et al. Reconstructed Single-Cell Fate Trajectories Define Lineage Plasticity Windows during Differentiation of Human PSC-Derived Distal Lung Progenitors. Cell Stem Cell 26, 593-608 e598, doi:10.1016/j.stem.2019.12.009 (2020).
10 Gage, B. K., Webber, T. D. & Kieffer, T. J. Initial cell seeding density influences pancreatic endocrine development during in vitro differentiation of human embryonic stem cells. PLoS One 8, e82076, doi:10.1371/journal.pone.0082076 (2013).
11 Ninomiya, H. et al. Improved efficiency of definitive endoderm induction from human induced pluripotent stem cells in feeder and serum-free culture system. In Vitro Cell Dev Biol Anim 51, 1-8, doi:10.1007/s11626-014-9801-y (2015).
12 Martyn, I., Brivanlou, A. H. & Siggia, E. D. A wave of WNT signaling balanced by secreted inhibitors controls primitive streak formation in micropattern colonies of human embryonic stem cells. Development 146, doi:10.1242/dev.172791 (2019).
13 Mahaddalkar, P. U. et al. Generation of pancreatic beta cells from CD177(+) anterior definitive endoderm. Nat Biotechnol 38, 1061-1072, doi:10.1038/s41587-020-0492-5 (2020).

1. A method of producing primitive streak cells for differentiation to definitive endoderm from a population of human pluripotent stem cells (hPSCs), the method comprising: differentiating a population of hPSCs to primitive streak cells in culture medium comprising Latrunculin A (LatA).

2. The method of claim 1, wherein the LatA is present in the culture medium on day 0.

3. The method of claim 1, wherein the culture medium further comprises Activin A.

4. The method of claim 1, wherein the culture medium further comprises a GSK3A/B inhibitor/canonical Wnt activator.

5. The method of claim 4, wherein the GSK3A/B inhibitor/canonical Wnt activator is, GSK Inhibitor IX, LiCl, WNT3 or CHIR99021, preferably CHIR99021.

6. The method of claim 1, wherein the culture medium further comprises a ROCK inhibitor.

7. The method of claim 6, wherein the ROCK1/2 inhibitor is Y-27632, fasudil, K-115 or AR-13503, GSK269962, H-1152, RKI-1447, HA-1100, or Thiazovivin, preferably Y-27632.

8. The method of claim 1, wherein the culture medium is mouse embryonic fibroblast free (MEF-free).

9. The method of claim 1, wherein the primitive streak cells are further differentiated into definitive endoderm cells.

10. The method of claim 9, wherein the differentiation into definitive endoderm is performed in the presence of Activin.

11. The method of claim 9, wherein the definitive endoderm cells are further differentiated into pancreatic progenitors.

12. The method of claim 9, wherein the definitive endoderm cells are further differentiated into lung progenitors.

13. The method of claim 9, wherein the definitive endoderm cells are further differentiated into liver progenitors.

14. The method of claim 9, wherein the definitive endoderm cells are further differentiated into intestine progenitors.

15. The method of claim 9, wherein the definitive endoderm cells are further differentiated into gastric progenitors.

16. The method of claim 9, wherein the definitive endoderm cells are further differentiated into esophageal progenitors.

17. The method of claim 9, wherein the definitive endoderm cells are further differentiated into thymus progenitors.

18. The method of claim 9, wherein the definitive endoderm cells are further differentiated into thyroid progenitors.

19. The population of primitive streak cells produced by the method of claim 1.

20. The population of definitive endoderm cells produced by the method of claim 9.

21. The population of progenitor cells produced by the method of claim 11.

22. The population of progenitor cells claim of 21, wherein the progenitor cells are pancreatic progenitor cells.
